(19) United States
(12) Patent Application Publication
(10) Pub. No.: US 2025/0026862 A1
(43) Pub. Date: Jan. 23, 2025

(54) A method for producing anionic saccharides

(71) Applicant: Kemira Oyj, Helsinki (FI)

(72) Inventor: Erkki Johannes METSÄLÄ, Helsinki (FI)

(21) Appl. No.: 18/712,740

(22) PCT Filed: Nov. 24, 2022
(86) PCT No.: PCT/FI2022/050785

§ 371 Date: May 23, 2024

(30) Foreign Application Priority Data
Dec. 2, 2021 (FI) 20216240

(51) Int. Cl. C08B 11/12; C07H 15/04; C08L 1/28

(52) U.S. Cl. CPC C08B 11/12; C07H 15/04; C08L 1/286

(57) ABSTRACT

The disclosure relates to a method for producing anionic saccharides by reacting saccharide with a compound having a conjugated electron withdrawing group via oxa-Michael addition reaction for producing a derivatized saccharide followed by anionizing the derivatized saccharide. The disclosure further relates to anionic saccharides and to use of the anionic saccharides, for example, as flocculants in waste water treatment, as an agent on enhanced oil recovery and as an agent in paper industry.

### TECHNICAL FIELD

The present disclosure generally relates to a method for producing anionic saccharides. The disclosure relates particularly, though not exclusively, to a method for producing anionic saccharides via oxa-Michael addition.

### BACKGROUND

This section illustrates useful background information without admission of any technique described herein representative of the state of the art.

Flocculation is a water treatment process where solids form larger clusters, or flocs, to be removed from water. Flocculants are substances that promote agglomeration of fine particles present in a solution, creating a floc, which then floats to the surface (flotation) or settles to the bottom (sedimentation). Flocculants can be organic or inorganic, and come in various charges, charge densities, molecular weights, and forms.

Organic polymeric flocculants are widely used, due to their ability to promote flocculation with a relatively low dosage. Although, their lack of biodegradability and the associated dispersion of potentially harmful monomers into water supplies is causing the focus to shift to biopolymers, which are more environmentally friendly. The problem with the biopolymers is they have a shorter shelf-life, and require a higher dosage than organic polymeric flocculants. To combat this, combined solutions are being developed, where synthetic polymers are grafted onto natural polymers, to create tailored flocculants for water treatment that deliver the optimum benefits of both.

Paper strengthening agents play an important role in the papermaking industry with the increase of secondary fibre application. The most commonly used paper strengthening agents are polyacrylamide, starch, chitosan, and other polymers. Starch is currently the most widely used dry-strength agent because of its relatively low price and high performance. Starch has an abundance of hydroxyl groups, which form hydrogen bonds with wood fibres to improve paper strength.

Starches are often modified with an anionic charge or with amphoteric starches to increase starch retention.

There is still a need for new methods for producing chemicals for water treatment and chemicals for papermaking.

### SUMMARY

The following presents a simplified summary of the features disclosed herein to provide a basic understanding of some exemplary aspects of the invention. This summary is not an extensive overview of the invention. It is not intended to identify key/critical elements of the invention or to delineate the scope of the invention. Its sole purpose is to present some concepts disclosed herein in a simplified form as a prelude to a more detailed description.

In a first aspect the present invention provides a method for producing anionic saccharides, comprising reacting a saccharide with a compound having a conjugated electron withdrawing group via oxa-Michael addition reaction in presence of a base or mixture of bases for producing a derivatized saccharide and anionizing the derivatized saccharide.

In a second aspect the present invention provides derivatized saccharides.

In a third aspect the present invention provides anionic saccharides.

In a fourth aspect the present invention provides a use of the anionic saccharide produced with the method of the present invention or the anionic saccharide of the present invention as a flocculant in industrial or municipal wastewater treatment, as an agent in enhanced oil recovery (EOR), as an agent in paper industry, as a dispersing agent, as an agent in mining or as an agent in textile industry.

It was surprisingly found that derivatized saccharides can be synthesized via oxa-Michael reaction in mild conditions. It was additionally found that by subjecting the derivatized saccharides to conventional anionization anionic saccharides can be produced.

With the method of the present invention synthesis of biobased and biodegradable precursors of anionic polymers can be prepared via a route avoiding epoxide chemistry. The method of the present invention provides a route to environmentally-friendly polymers and anionic polymers, such as anionic saccharides.

It was also found that the produced derivatized saccharides and anionic saccharides can be used in variety of applications, for example, as flocculants, such as a biobased flocculant, in wastewater treatment, as an agent in enhanced oil recovery, in mining, in textile, as dispersing agent and as an agent in paper industry.

The appended claims define the scope of protection.

### BRIEF DESCRIPTION OF THE FIGURES

FIG. 1 shows ¹HNMR profile of a reaction mixture comprising glucose (monohydrate), acrylic acid and a-d-glucose (C6-OH) derivatized with acrylic acid.

FIG. 2 shows ¹³CNMR profile of a reaction mixture comprising glucose (monohydrate), acrylic acid and a-d-glucose (C6-OH) derivatized acrylic acid.

### DETAILED DESCRIPTION

In first aspect the present invention provides a method for producing anionic saccharides. More particularly, the present invention provides a method for producing anionic saccharides comprising reacting a saccharide with a compound having a conjugated electron withdrawing group via oxa-Michael addition reaction in presence of a base or mixture of bases for producing a derivatized saccharide, and anionizing the derivatized saccharide for producing the anionic saccharide.

By “Oxa-Michael addition reaction” is meant a conjugate nucleophilic addition reaction involving O-nucleophiles (Michael donors) and Michael acceptors.

By “an electron withdrawing group” is meant a group that draws electrons away from reaction center. Examples of electron withdrawing groups are halogens (F, CI), nitriles (CN), carbonyls (RCOR′) and nitro groups (NO₂).

By “a compound having a conjugated electron withdrawing group” is meant a compound that has an electron withdrawing group that is conjugated; for example CH2═CH—C═O (i.e. double bond-single bond-double bond) and CH2═CH—NR2 (i.e. double bond-single bond).

In one embodiment the carbon-carbon double bond of the compound having a conjugated electron withdrawing group is terminal i.e. primary.

In one embodiment the carbon-carbon double bond of the compound having a conjugated electron withdrawing group is intramolecular.

In a preferred embodiment the carbon-carbon double bond of the compound having a conjugated electron withdrawing group is terminal i.e. primary.

In one embodiment a saccharide is reacted with a compound having a conjugated electron withdrawing group via oxa-Michael addition reaction by bringing the saccharide into contact with the compound having a conjugated electron withdrawing group in presence of a base or a mixture of bases.

The derivatized saccharide is formed via the oxa-Michael addition reaction. In oxa-Michael addition reaction carbon-carbon double bond of a compound having a conjugated electron withdrawing group reacts with hydroxyl group of the saccharide, thus, forming the derivatized saccharide.

In one embodiment one or more hydroxyl groups of the saccharide react with one or more of the compound having a conjugated electron withdrawing group, thus forming derivatized saccharide.

The derivatized saccharide can be anionized with any suitable method in the art. The derivatized saccharide can be treated with aqueous medium such as water, at least one base or a mixture thereof. When the saccharide derivatized with acrylic acid is treated with aqueous medium, at least one base or a mixture thereof, the hydroxyl group of the acrylic acid moiety is deprotonated, thus forming an anionic saccharide.

It is known in the art that when carboxylic acids dissolve, hydrogen (H) dissociate, thus forming O⁻ charge. It is also known in the art that when potassium (K), sodium (Na) or lithium (Li) salts of carboxylic acid dissolve, K, Na or Li dissociate, thus forming O⁻ charge.

In one embodiment the derivatized saccharide is recovered prior the anionization.

In one embodiment the recovered derivatized saccharide is anionized.

In one embodiment the derivatized saccharide is recovered prior anionization and the recovered derivatized saccharide is anionized.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as β-cyclodextrin or a mixture thereof, preferably the polysaccharide comprises starch, glycogen, galactogen, chitosan, chitin, guar gum, pectin, dextran, α-glucan, cyclodextrin such as β-cyclodextrin or a mixture thereof, more preferably the polysaccharide is selected from starch, glycogen, galactogen, chitosan, chitin, guar gum, pectin, dextran, α-glucan, cyclodextrin such as β-cyclodextrin, and a mixture thereof.

In one embodiment the saccharide is glucose, starch or cellulose.

In one embodiment the compound having a conjugated electron withdrawing group (EWG) comprises compounds having formula R1R2C═CR3-EWG, wherein R1, R2 and R3 represents independently a hydrogen atom or an alkyl chain having 1 to 20 carbon atoms; and EWG represents an electron withdrawing group preferably a carboxyl, a sulphonate or a phosphate group.

In one embodiment the R1, R2 and R3 represent independently a hydrogen atom or an alkyl chain having 1 to 10 carbon atoms.

In one embodiment the compound having a conjugated electron withdrawing group is in form of a salt, such as a sodium (Na) salt, potassium (K) salt or lithium (Li) salt.

In one embodiment the electron withdrawing group is selected from carboxylic acids, sulphonates or phosphates.

In one embodiment the compound having a conjugated electron withdrawing group is anionic.

In one embodiment the compound having a conjugated electron withdrawing group is selected from one of the following compounds I-VI

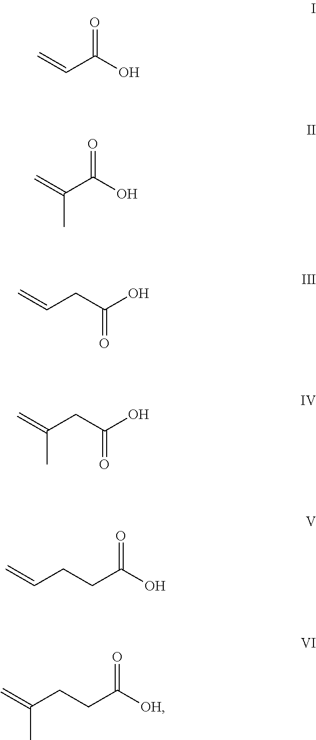

or a sodium (Na), potassium (K) or lithium (Li) salt thereof.

In one embodiment the compound having a conjugated electron withdrawing group is compound I or II

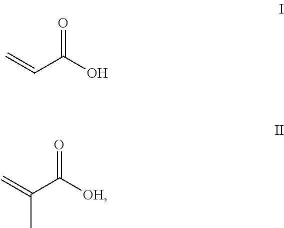

or a sodium (Na), potassium (K) or lithium (Li) salt thereof.

In one embodiment in the method for producing anionic saccharides mole ratio of the saccharide to the compound having a conjugated electron withdrawing group is 1:1.

In one embodiment the base present in the method for producing anionic saccharides comprises NaOH, KOH, potassium tert-butoxide (tBUOK), diazabicycloundecene (DBU), MeONa or a mixture thereof.

In one embodiment temperature in the oxa-Michael addition reaction is 15° C.-30° C., preferably 18° C.-25° C.

In one embodiment pressure in the oxa-Michael addition reaction is prevailing atmospheric pressure, preferably normal atmospheric pressure, more preferably about 1 bar.

In one embodiment a liquid medium is present in the oxa-Michael addition reaction, i.e. the oxa-Michael addition reaction takes place in a liquid medium. Preferably the liquid medium is a polar protic solvent or a mixture of polar protic solvents, more preferably the liquid medium is water.

In one embodiment the saccharide, the compound having a conjugated electron withdrawing group and the base or the mixture of bases are mixed during the oxa-Michel addition reaction.

In one embodiment the saccharide, the compound having a conjugated electron withdrawing group and the base or the mixture of bases are mixed during the oxa-Michel addition reaction from 1 min to 24 hours, or at least 12 hours, such as from 12 hours to 24 hours; or less than 12 hours, such as from 1 min to 11 hours 45 min.

During the oxa-Michael addition reaction a reaction mixture is formed comprising the saccharide, the compound having a conjugated electron withdrawing group, the base or the mixture of bases and a derivatized saccharide.

In one embodiment the reaction mixture is mixed during the oxa-Michel addition reaction from 1 min to 24 hours, or at least 12 hours, such as from 12 hours to 24 hours; or less than 12 hours, such as from 1 min to 11 hours 45 min.

Required mixing time i.e. reaction time depends on the compound having a conjugated electron withdrawing group. The higher molecular weight of the compound having a conjugated electron withdrawing group is, the longer the reaction time is. Additionally, the stronger polar strength of the compound having a conjugated electron withdrawing group is the faster is the reaction.

In one embodiment the anionization reaction takes place in an inert liquid medium.

In one embodiment the anionized saccharide is subjected to an additional anionization.

One or more of the above described embodiments may be combined.

In a second aspect, the present invention provides a derivatized saccharide. More particularly the present invention provides a derivatized saccharide, wherein the saccharide is derivatized with a compound having a conjugated electron withdrawing group.

In one embodiment the derivatized saccharide is produced with the method of the present invention via oxa-Michael addition reaction of a saccharide and a compound having a conjugated electron withdrawing group.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose, sucrose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as β-cyclodextrin or a mixture thereof, preferably the polysaccharide comprises starch, glycogen, galactogen, chitosan, chitin, guar gum, pectin, dextran, α-glucan, cyclodextrin such as β-cyclodextrin or a mixture thereof, more preferably the polysaccharide is selected from starch, glycogen, galactogen, chitosan, chitin, guar gum, pectin, dextran, α-glucan, cyclodextrin such as β-cyclodextrin, and a mixture thereof.

In one embodiment the saccharide is glucose, starch or cellulose.

In one embodiment the compound having a conjugated electron withdrawing group (EWG) comprises compounds having formula R1R2C═CR3-EWG, wherein R1, R2 and R3 represents independently a hydrogen atom or an alkyl chain having 1 to 20 carbon atoms; and EWG represents an electron withdrawing group, preferably a carboxyl, a sulphonate or a phosphate group.

In one embodiment the R1, R2 and R3 represents independently a hydrogen atom or an alkyl chain having 1 to 10 carbon atoms

In one embodiment the compound having a conjugated electron withdrawing group is anionic.

In one embodiment the compound having a conjugated electron withdrawing group is selected from one of the following compounds I-VI

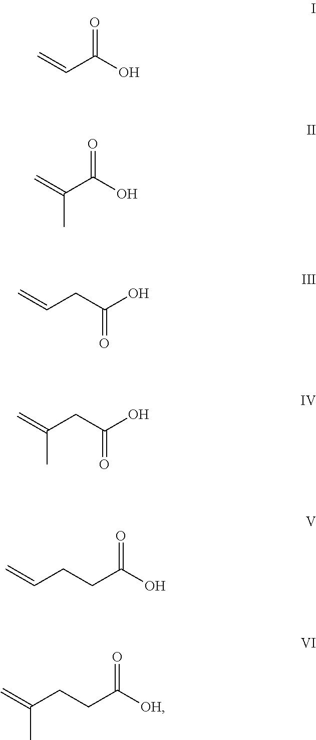

or a sodium (Na), potassium (K) or lithium (Li) salt thereof.

In one embodiment the compound having a conjugated electron withdrawing group is compound I or II

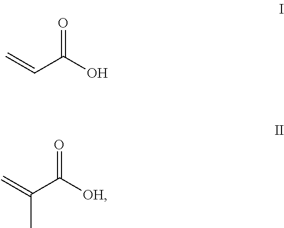

or a sodium (Na), potassium (K) or lithium (Li) salt thereof.

In one embodiment the derivatized saccharide is glucose, starch or cellulose derivatized with compound I or II

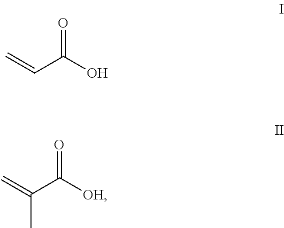

or a sodium (Na), potassium (K) or lithium (Li) salt thereof.

In one embodiment the derivatized saccharide has the following formula VII

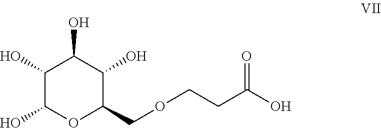 [SMILES: O=C(O)CCOC[C@H]1O[C@H](O)[C@H](O)[C@@H](O)[C@@H]1O]

In a third aspect the present invention provides an anionic saccharide. The anionic saccharide comprises anionic saccharides having the following formula VIII

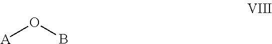

wherein A represents a monosaccharide, a disaccharide, an oligosaccharide or a polysaccharide; B represents an anionic compound having formula R1R2C—CR4R3-EWG, wherein R1, R2, R3 and R4 represents independently a hydrogen atom or an alkyl chain having 1 to 20 carbon atoms; EWG represents an electron withdrawing group, preferably carboxyl, a sulphonate or a phosphate group; and O having a bound to A is an oxygen atom of the monosaccharide, the disaccharide, the oligosaccharide or the polysaccharide.

In one embodiment the R1, R2, R3 and R4 represents independently a hydrogen atom or an alkyl chain having 1 to 10 carbon atoms.

In one embodiment the electron withdrawing group is selected from carboxylic acids, sulphonates or phosphates.

In one embodiment the saccharide comprises monosaccharides, disaccharides, oligosaccharides, polysaccharides or a mixture thereof.

In one embodiment the monosaccharide comprises glucose, fructose, galactose, mannose or a mixture thereof.

In one embodiment the disaccharide comprises sucrose, lactose, maltose, sucrose or a mixture thereof.

In one embodiment the oligosaccharide comprises glycan, raffinose, maltodextrin, cellodextrin or a mixture thereof.

In one embodiment the polysaccharide comprises starch, glycogen, galactogen, cellulose, chitosan, chitin, guar gum, pectin, dextran, α-glucan, cyclodextrin such as β-cyclodextrin or a mixture thereof, preferably the polysaccharide comprises starch, glycogen, galactogen, chitosan, chitin, guar gum, pectin, dextran, α-glucan, cyclodextrin such as β-cyclodextrin or a mixture thereof, more preferably the polysaccharide is selected from starch, glycogen, galactogen, chitosan, chitin, guar gum, pectin, dextran, α-glucan, cyclodextrin such as β-cyclodextrin, and a mixture thereof.

In one embodiment the anionic saccharide comprises anionic saccharides having the following formulas IX-XIV

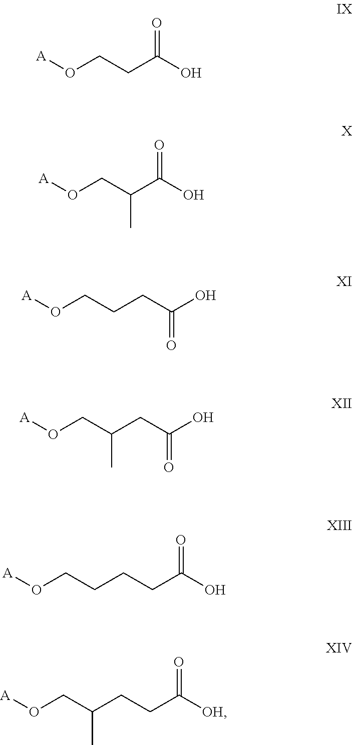

or a sodium (Na), potassium (K) or lithium (Li) salt thereof, wherein A represents a monosaccharide, a disaccharide, an oligosaccharide or a polysaccharide and O having a bound to A is an oxygen atom of the monosaccharide, the disaccharide, the oligosaccharide or the polysaccharide.

In one embodiment the anionic saccharide has the following formula IX or X

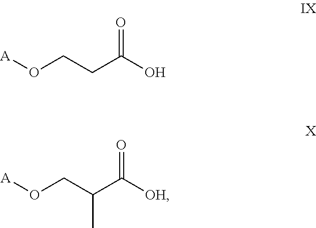

or a sodium (Na), potassium (K) or lithium (Li) salt thereof.

In one embodiment the anionic saccharide has the following formula IX or X

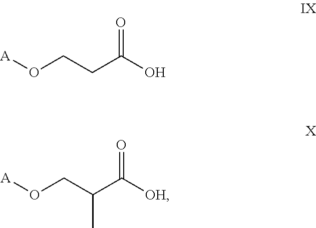

or a sodium (Na), potassium (K) or lithium (Li) salt thereof,
wherein A represents glucose, starch or cellulose and O having a bound to A is an oxygen atom of the glucose, starch or the cellulose.

In one embodiment the anionic saccharide is produced with the method of the present invention.

One or more of the above embodiments can be combined.

In a fourth aspect the present invention provides use of the anionic saccharide.

More particularly, the present invention provides use of the anionic saccharide produced with the method of the present invention or the anionic saccharide of the present invention as a flocculant in industrial or municipal wastewater treatment, as an agent in enhanced oil recovery (EOR), as an agent in paper industry, as a dispersing agent, as an agent in mining or as an agent in textile industry.

### EXAMPLES

### Example 1 According to the Present Invention

### Materials

In table 1 is disclosed used compounds and properties of the compounds.

TABLE 1
Compounds and properties the compounds.

| Starting | CAS | M | n | m | mp | bp | Density | Volume | Refractive |
|---|---|---|---|---|---|---|---|---|---|
| materials | number | (g/mol) | (mol) | (g) | (° C.) | (° C.) | (g/ml) | (ml) | index |
| Glucose | 14431-43-7 | 198.17 | 0.0252 | 5.00 | 83 | | | | |
| (monohydrate) | | | | | | | | | |
| Acrylic acid | 79-10-7 | 72.06 | 0.0250 | 31.8 | | 141 | 1.051 | 1.71 | 1.42 |
| NaOH | 1310-73-2 | 40.00 | 0.0010 | 0.04 | | | 2.13 | | |

Synthesis of a-d-glucose (C6-OH) derivatized with acrylic acid

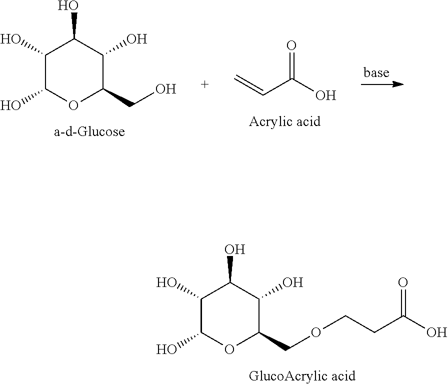

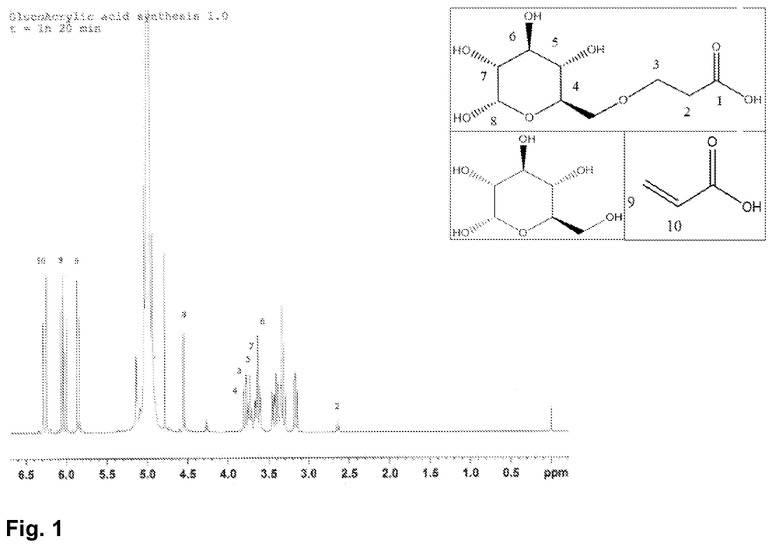

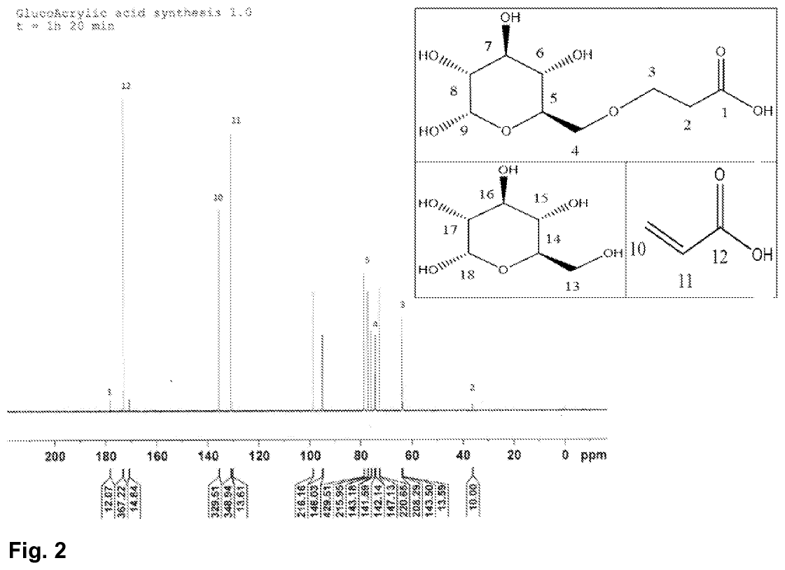

Glucose (monohydrate) was dissolved into 7 ml of pure water (MQ) followed by adding 0.04 g of NaOH. Reaction mixture was mixed with magnetic stirrer and 1.71 ml of acrylic acid was added, during mixing, dropwise during five minutes to the reaction mixture followed by mixing the reaction mixture for twelve hours. 1HNMR and 13CNMR profiles of the reaction mixture were analysed and molecular mass was measured after the 12 hours. The NMR profiles are shown in FIGS. 1 and 2.

### Characterisation

Molecular mass of the obtained a-d-glucose (C6-OH) derivatized with acrylic acid is 252.22 g/mol.

FIG. 1 shows 1HNMR profile of the reaction mixture comprising the starting materials glucose (monohydrate) and acrylic acid and the product a-d-glucose (C6-OH) derivatized with acrylic acid.

In the FIG. 1 carbons of the starting materials and the product are numbered. Chemical shifts of the protons having a bound to the numbered carbons are marked to the 1HNMR profile. Chemical shifts of protons of the glucose are not marked to the 1HNMR profile.

FIG. 2 shows 13CNMR profile of a reaction mixture comprising the starting materials glucose (monohydrate) and acrylic acid and the product a-d-glucose (C6-OH) derivatized with acrylic acid.

In the FIG. 2 carbons of the starting materials and the product are numbered. Chemical shifts of the carbons are marked to the 13CNMR profile. Chemical shifts of carbons of the glucose and the glucose unit are not marked to the 13CNMR profile except chemical shifts of the carbons 4, 5 and 18.

Based on the measured molecular mass and the 1HNMR and 13CNMR profiles a derivatized saccharide, namely, a-d-glucose (C6-OH) derivatized with acrylic acid is produced with the method of the present invention.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A method for producing anionic saccharides, comprising reacting saccharide with a compound having a conjugated electron withdrawing group via oxa-Michael addition reaction in presence of a base or mixture of bases for producing a derivatized saccharide and anionizing the derivatized saccharide, wherein the compound having a conjugated electron withdrawing group is selected from one of the following compounds III-VI 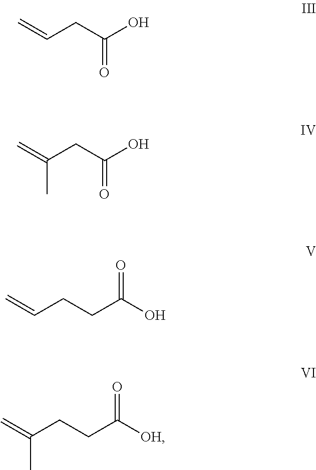
or a sodium, potassium or lithium salt thereof.

2. The method according to claim 1, wherein one or more catalysts are present in the oxa-Michael addition reaction and/or in the anionization.

3. The method according to claim 1, wherein the derivatized saccharide is recovered prior anionization and the recovered derivatized saccharide is anionized.

4. The method according to claim 1, wherein the saccharide comprises monosaccharides, disaccharides, oligosaccharides, polysaccharides or a mixture thereof.

5. A derivatized saccharide comprising glucose, starch or cellulose derivatized with a compound selected from the compounds III-VI 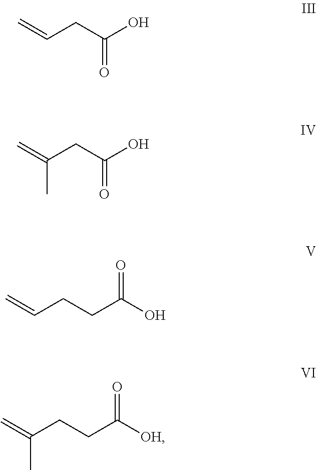
or a sodium, potassium or lithium salt thereof.

6. The derivatized saccharide according to claim 5, wherein the derivatized saccharide has the following formula VII

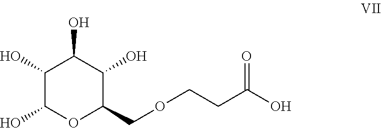 [SMILES: O=C(O)CCOC[C@H]1O[C@H](O)[C@H](O)[C@@H](O)[C@@H]1O]

7. An anionic saccharide comprising anionic saccharides having formulas XI-XIV 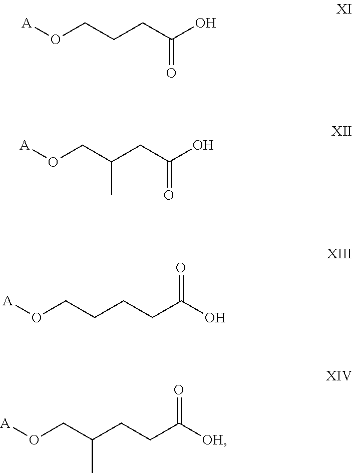
or a sodium, potassium or lithium salt thereof; wherein A represents a monosaccharide, a disaccharide, an oligosaccharide or a polysaccharide; and O having a bound to A is an oxygen atom of the monosaccharide, the disaccharide, the oligosaccharide or the polysaccharide.

8. An anionic saccharide according to claim 7, wherein the A represents glucose, starch or cellulose.

9. (canceled)

10. The method according to claim 2, wherein the derivatized saccharide is recovered prior anionization and the recovered derivatized saccharide is anionized.

11. The method according to claim 2, wherein the saccharide comprises monosaccharides, disaccharides, oligosaccharides, polysaccharides or a mixture thereof.

12. The method according to claim 3, wherein the saccharide comprises monosaccharides, disaccharides, oligosaccharides, polysaccharides or a mixture thereof.
